(19) United States
(12) Patent Application Publication
(10) Pub. No.: US 2005/0075972 A1
(43) Pub. Date: Apr. 7, 2005

(54) Risk assessment system and method of adjusting standard

(76) Inventor: Thomas James Doyle, Arthur (CA)

Correspondence Address: DARYL W SCHNURR;MILLER THOMSON LLP, PO BOX 578, SUITE 700, 22 FREDERICK STREET, KITCHENER, ON, N2G 4A2, CA

(21) Appl. No.: 10/957,660

(22) Filed: Oct. 5, 2004

Related U.S. Application Data
(63) Continuation-in-part of application No. 10/678,117, filed on Oct. 6, 2003.

(51) Int. Cl. G06F 17/60; A61B 13/00

(52) U.S. Cl. 705/38

(57) ABSTRACT

A method and system of assessing the risk of using industrial equipment to a user allows inputs to be made to a program electronically. When the appropriate inputs have been entered for a plurality of risk factors relating to the industrial equipment, the program will estimate a net risk of injury to the user based on the information inputted and based on evaluation data within the program. The risk evaluation is determined automatically by the program.

This is a continuation-in-part application of Application Serial No. 10/678,117 filed Oct. 6, 2003.

### BACKGROUND OF THE INVENTION

1. Field of Invention

This invention relates to a system and method for assessing the risk of using industrial equipment to a user thereof. More particularly, the system enables inputs to be made with respect to various risk factors relating to the equipment. The system summarizes the inputs and produces a risk evaluation for the industrial equipment being evaluated.

2. Description of the Prior Art

Original equipment manufacturers and users of that equipment have found it necessary to evaluate the risk of injury to users of that equipment. In some jurisdictions, risk evaluation of industrial equipment is required by law before the equipment is put into use. Previously, risk assessment has been determined by experienced risk evaluators. Since the assessment is made by a particular evaluator, the assessment is extremely subjective as the personal experience of the person conducting the evaluation often determines the level of risk assessment. More recently, a paper form or forms are required to be completed in order to conduct the risk assessment. These forms can sometimes be twenty pages or more in length. When a risk assessment is determined by completing such form, the assessment is more objective than a verbal assessment. However, the paper form takes a great deal of time to complete and particular risk factors may not be covered where those factors specifically apply to the particular piece of equipment being evaluated. Also, the completion of the paper form is still somewhat subjective and it is extremely time consuming to determine the result of the risk evaluation. Also, with the paper form, individual risk values are not applied to each of the risk factors making it more difficult and more time consuming to consistently determine the end results.

### SUMMARY OF THE INVENTION

It is an object of the present invention to provide a system and method for risk assessment of industrial equipment where information can be inputted into the system for those risk factors that apply to the industrial equipment, the system having an inherent valuation for each input and the system automatically determining a risk evaluation for the industrial equipment based on the information inputted. It is a further object of the present invention to produce a written risk evaluation from an electronic evaluation which can be stored and updated electronically as desired.

A method of assessing the risk of using industrial equipment to a user thereof by preparing a risk evaluation using a program, said method comprising:
(a) choosing a safeguard performance standard for the risk evaluation;
(b) inputting to said program information relating to a plurality of risk factors;
(c) causing the program to estimate a net risk of injury to said user of the industrial equipment based on the information and based on evaluation data within the program;
(d) the program producing a risk evaluation for the industrial equipment.

A risk assessment system for use with a computer, said system assessing the risk of injury to a user from industrial equipment by preparing a risk evaluation. The system comprises:
(a) a risk assessment system for use with a computer, the system assessing the risk of injury to a user from industrial equipment by preparing a risk evaluation, the system comprising:
   (i) a range of pre-determined risk values for each of a plurality of potential risk factors for which inputs are available for said industrial equipment;
   (ii) the system displaying each of the risk factors on demand and a range of inputs for a level of risk for each of the risk factors for which inputs are available;
   (iii) the system accepting an input for each risk factor for which inputs are available;
   (iv) the system determining a net value for all of the inputs and producing an assessment of risk for the industrial equipment;
   (v) a selection for at least one of a safeguard performance standard option for said risk evaluation.

Preferably, the system has at least one of a choice of safeguard performance standards and an end user editing feature.

### BRIEF DESCRIPTION OF THE DRAWINGS

FIG. 1 is a general block diagram of the system of the present invention;

FIG. 2 is a more detailed block diagram of the system;

FIG. 3 is a top view of a form of risk assessment as it appears on a computer screen;

FIG. 4 is a top view of a point of operation pop-up menu of the form of risk assessment with the details of risk estimation shown in a pop-up menu;.

FIG. 5 is a top view of a point of operation pop-up menu of FIG. 4 with a level of risk responses to severity of potential injury caused by hazard;

FIG. 6 is a top view of a point of operation pop-up menu with a level of risk responses partially shown for a risk reduction method based on mechanical safety devices;

FIG. 7 is a top view of a point of operation pop-up menu with a level of risk responses shown for one characteristic of the facility;

FIG. 8 is a top view of a form of point of operation having a drop down menu for a choice of safeguard performance standard;

FIG. 9 is a top view of an assessment form having a space for a performance standard designation;

FIG. 10 is a block diagram of a risk assessment system having a choice of safeguard performance standard; and

FIG. 11 is a top view of a point of operation showing an editing space for a task hazard document.

### DETAILED DESCRIPTION OF A PREFERRED EMBODIMENT

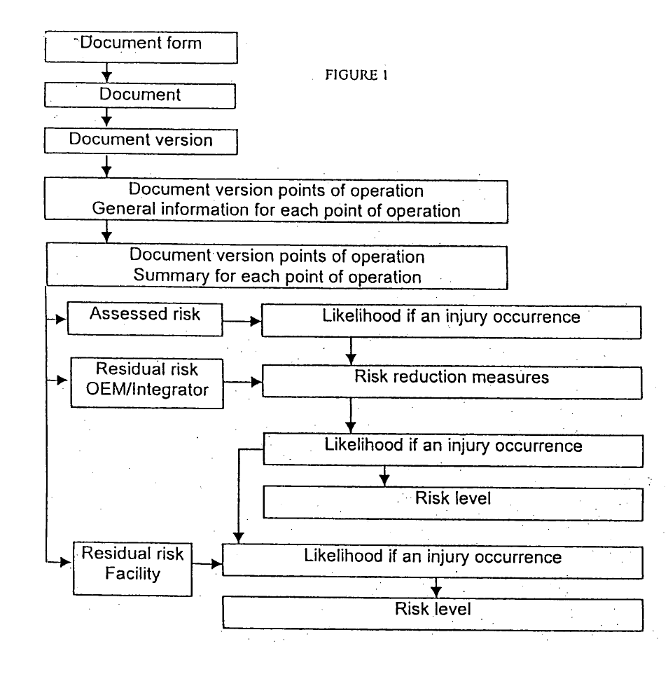

In FIG. 1, there is shown a block diagram of the risk assessment system of the present invention. The risk assessment document initially requires information concerning the owner of the equipment. That information can be entered into a separate form and transferred onto the risk assessment document so that when the owner is the same, the information is inputted onto the separate form only the first time. If there are no changes in the information, the information can be transferred onto the risk assessment form as often as required. A description of the equipment and machinery sufficient to identify it is also required. Next, information is inputted onto the risk assessment form identifying the points of operation of the equipment or machine being evaluated. There is a separate risk assessment conducted for each point of operation. The risk assessment is then conducted for the three risk factors, assessed risk, residual risk for the original equipment manufacturer/integrator and residual risk resulting from the facility in which the equipment is located. For the assessed risk, an assumption is made that the equipment and machinery does not have any guards or other protection means. The risk factor, probability of hazard occurrence, is assumed to be 100%. Inputs are made with respect to the assessed risk for the likelihood of an injury occurrence. No inputs are available for the probability of hazard occurrence, but inputs are available for the three risk factors that make up the probability of injury occurrence. These three risk factors are severity of potential injury caused by hazard, frequency of exposure to hazard, and possibility of hazard avoidance. Then, inputs are made with respect to risk reduction measures by the original equipment manufacturer (OEM) Integrator and the likelihood of an injury occurrence is determined in light of those risk reduction factors. Next, the risk assessment determines the residual risk determined by the characteristics of the facility in which the equipment is to be used. The likelihood of an injury occurrence is again estimated. The system automatically assigns a value for each level of risk inputted into the system. Those values are either positive or negative depending on the level of risk and the type of risk that is being determined. For example, a level of risk that increases risk can be positive and a level of risk with respect to a risk factor that reduces risk is negative. The system totals the various inputs and produces a risk evaluation for the equipment or machinery.

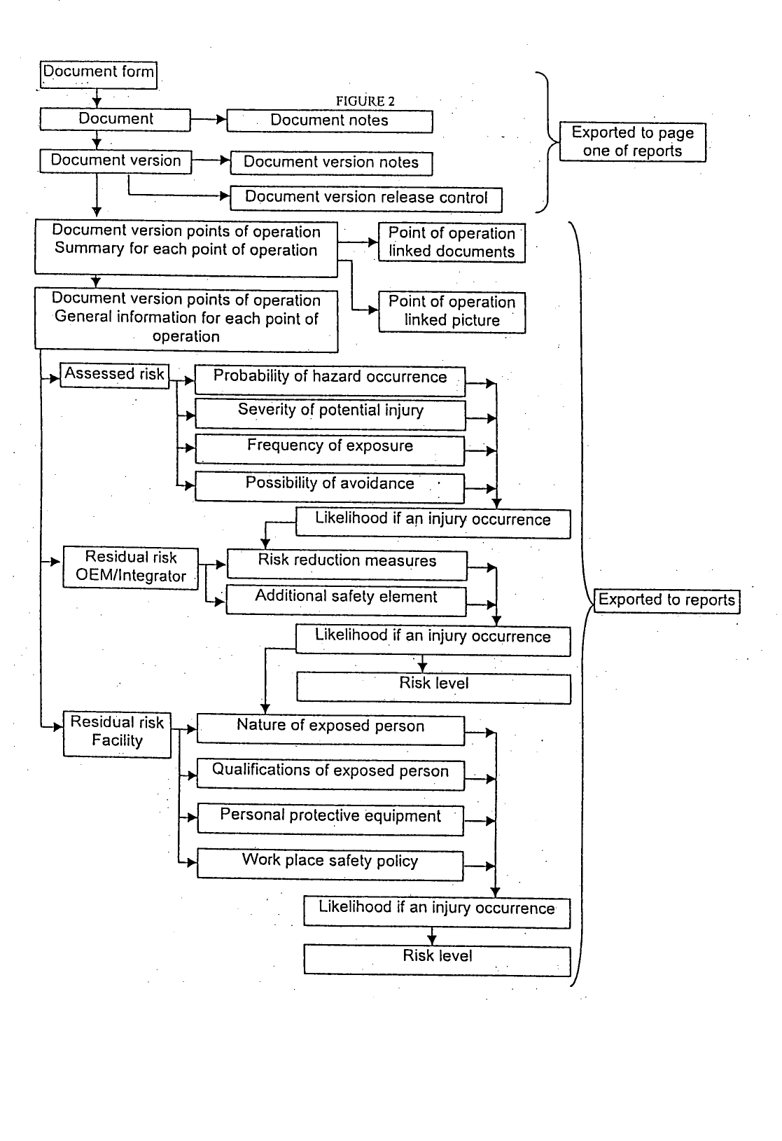

In FIG. 2, there is shown a more detailed block diagram of the risk assessment system of the present invention. The system allows documents to be linked to a particular piece of industrial equipment or machinery. For example, an incident report of a previous accident or injury associated with the machinery or equipment can be kept as part of the records. Similarly, other documents or even pictures or photographs can be linked to the risk evaluation.

Under assessed risk, levels of risk are inputted for each of three risk factors that together make up the probability of injury occurrence. The three risk factors are severity of potential injury, frequency of exposure and possibility of avoidance. These three risk factors are considered on the basis that the equipment or machinery has no guards or safety features in place. Preferably, responses to all three factors are mandatory. The probability of injury occurrence from the three risk factors is then determined by the system.

Next, the residual risk reduction by the original equipment manufacturer (OEM/Integrator) is considered. Risk reduction measures that are mechanical and risk reduction measures that are passive are considered. Only one of the two factors can be chosen for each point of operation of the machinery or equipment. In other words, if mechanical risk reduction factors are present, then passive risk reduction measures cannot be considered and vice versa.

The residual risk of the machinery or equipment based on the safety characteristics of the facility are then considered. Factors such as the nature of the exposed person, qualifications of the exposed person, personal protective equipment and workplace safety policy are considered and inputs as to the level of risk or presence of the safety features are made. While there is more than one input for each risk factor, it is not possible to choose more than one input for each risk factor based upon the safety characteristics of the facility. For example, personal protective equipment could have one input for safety goggles and for safety footwear and another input for goggles, footwear and gloves. When one of the two inputs is chosen, no further inputs can be made for that risk factor. The likelihood of an injury occurrence from each of the three main risk factors is determined and a risk evaluation is made by totalling the likelihood of an injury occurrence from the three factors.

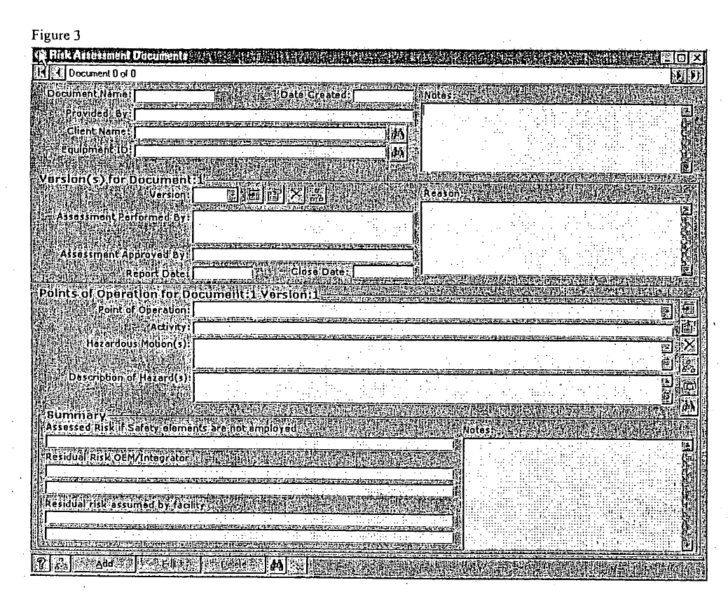

In FIG. 3, it can be seen that the form has an identification section where the characteristics of the equipment can be set out, a risk estimation section and a risk reduction section. The form also sets out residual risks and characteristics of the facility in which the equipment is installed or is to be installed. It can be seen that notes can be added to the risk assessment document in several locations. After the initial information has been inputted, inputs can be made as to the level of risk with respect to the assessed risk and the residual risk reduction portion of the document.

The system of the present invention is a software program that is preferably used on a computer. The program enables a consistent risk assessment to be conducted for industrial machinery and equipment. Pop-up menus set out a plurality of risk factors for estimating risk as well as a plurality of risk factors for reducing risk. When a particular risk factor is displayed on a monitor, a further pop-up menu discloses a range of inputs for that particular risk factor from minor to major. A user selects a particular risk factor and then inputs an appropriate input for that particular risk factor into the computer. Preferably, all of the risk factors relating to risk estimation require an input. For some industrial machines or equipment, some of the risk factors for reducing risk will be inapplicable and no input will be necessary for those risk factors in most instances. Machinery and equipment are considered to be synonymous in this application.

The system of the present invention presents a form for risk assessment on the monitor. The form provides a guide that can be used to obtain a risk evaluation for a particular piece of industrial equipment. The form can be completed for one piece of industrial equipment and preferably printed from the computer and saved electronically. The form can then be used to obtain a risk evaluation for another piece of industrial equipment. When the appropriate inputs have been made for a particular piece of industrial equipment, the system can automatically summarize the inputs to produce a risk evaluation for that equipment. The system contains predetermined values of risk for each level of risk in each range of risk for all of the risk factors set out in the pop-up menus. The level of risk in each range is chosen. These values are then broken down among the range of inputs for each risk factor. The system preferably requires inputs for all of the risk factors relating to estimation of risk but does not require an input for all of the remaining risk factors.

In FIG. 3, there is shown an embodiment of the form has a place for the client name and the identification of a particular piece of equipment. Some equipment may have two or more areas where hazardous activity will occur when the equipment is operated. Each hazardous area should be evaluated in a separate risk evaluation. The report has three sections relating to risk evaluation. These are risk estimation, risk reduction and characteristics of the facility from a risk perspective. Each area of the report relating to risk evaluation can be broken down further, preferably by pop-up menus. Alternatively, the breakdown of each section of risk evaluation can be part of the form without a pop-up menu. Each section of risk evaluation has a plurality of risk factors. Each risk factor has a range of risk, being inputs or responses that are used to evaluate that particular risk factor. The range risk for each risk factor allows the user to choose a level of risk for each risk factor. The levels of risk are preferably available to a user of the system by way of a pop-up menu.

In FIG. 4, there is shown a point of operation form with risk estimation on a pop-up menu of the form. It can be seen that an entry has been made as to the level of risk for severity of potential injury caused by the hazard as being “fatal—immediate or subsequent death”. Similarly, input as to level of risk has been made for assessed risk if safety elements are not employed as “possible-injury is likely”.

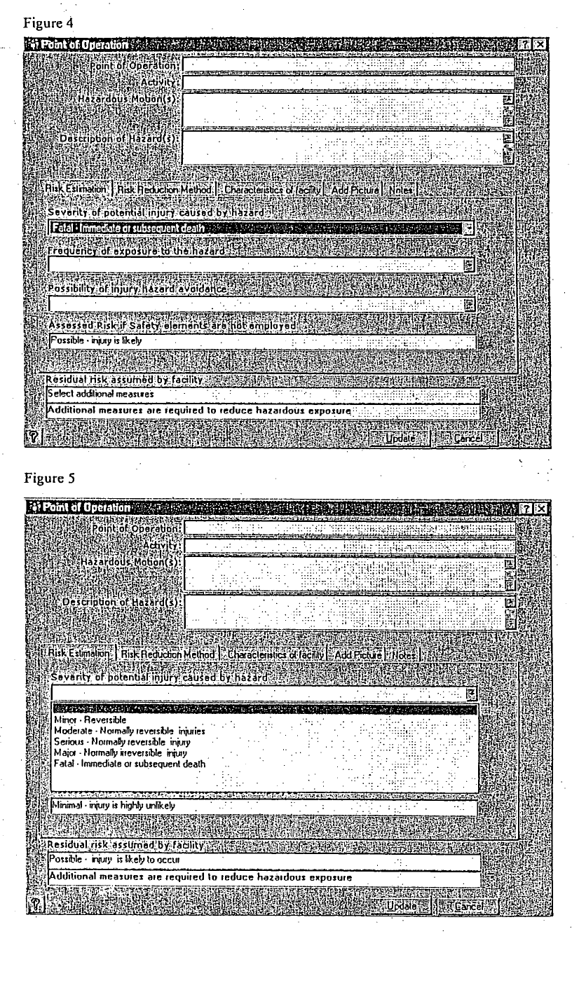

In FIG. 5, point of operation shows a pop-up menu for risk estimation for the severity of potential injury caused by the hazard ranging from minor through moderate, serious, major and fatal. Any one of the inputs can be selected for the severity of potential injury caused by the hazard and the system will then apply a risk value for the level of risk selected.

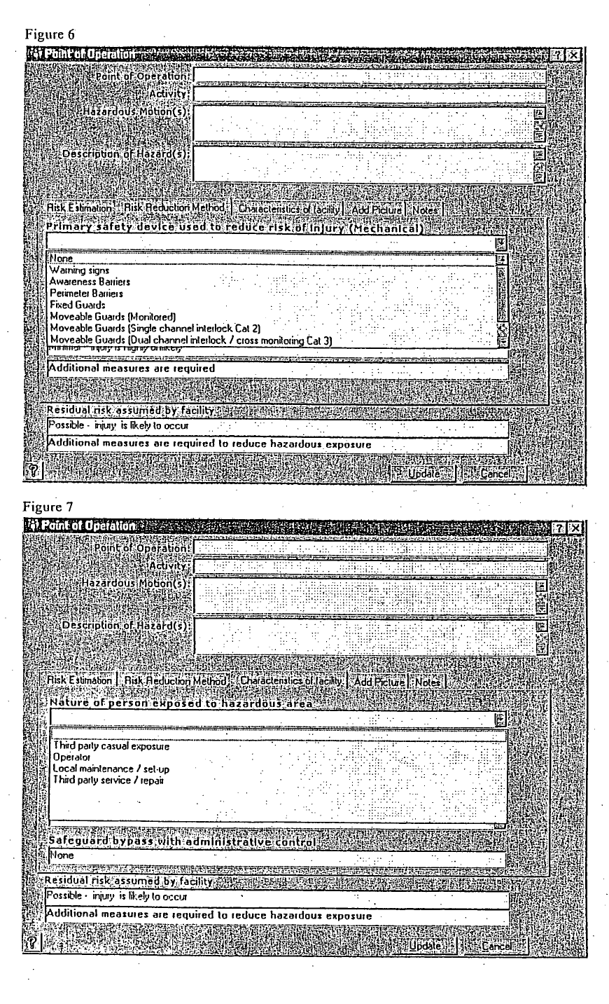

In FIG. 6, the point of operation form shown has the pop-up menu for risk reduction method for a mechanical primary safety device. Not all of the available inputs are shown and more than one input can be made in response to this risk factor.

In FIG. 7, the point of operation form shown has a pop-up menu for the characteristics of the facility for the nature of person exposed to the hazardous area. More than one input can be made from the list shown, which is third party casual exposure, operator, local maintenance/set-up and third party service/repair.

### EXAMPLE NUMBER 1

### Automatic Condition Assessments

### Risk Assessment

### Hazard Identification, Guarding Design, & Risk Reduction Methods, for Industrial Machinery

Program Features

Program Origin

The motivation behind the development of the risk assessment program was the lack of consistency in industry, as a whole, regarding industrial machinery safety. The need for industry to evaluate industrial machinery hazards and their effect on the health and safety of the employees in industrial manufacturing environments has been driven mainly by government regulation. As is the case with many regulations, the objective is defined, but the means that will enable the objective to be achieved is not.

In this time of global marketing, harmonization of products, and freedom of the workforce to move from continent to continent, some utilities will be required that will provide a safe, fair, and competitive environment for machinery manufacturers, production facilities, and end users. This program provides a key ingredient in the harmonization process.

General

This program may be used as an individual effort or prepared by a group. If an individual performs the analysis, it would be in the individual's best interest to obtain the opinion of others in order to provide different perspectives. Obviously designers play a key role in safety analyses but they should understand that not only the design and general hazards contribute to the risk of injury. It is important, when analyzing potential risk of injury, to include users, assemblers, repair and maintenance personnel, someone knowledgeable and trained in conducting safety analyses, and legal counsel, to gain valuable insight to uses, potential misuse, and hazards that may be overlooked by an individual. A team approach will usually produce a better result.

This risk assessment form is a tool that can be employed by manufacturers and end users of industrial machinery. It should be seen as an enhancement to sound design using experience-based approaches such as codes of practice rather than a substitute for such approaches. This form is to be utilized as a guide, not an expert system. If the user inputs poor and/or incomplete data, the resultant evaluation will be poor and/or incomplete. The results of this form should be considered subjective. Much of the data input to the risk assessment form are subjective decisions based on good engineering judgment. Therefore, the results from the analysis are no more objective than the input data. This form helps provide complete information organized in a usable format from which engineering decisions can be made based upon good collective judgment.

The program has been created such that the user is guided through the process of performing each stage of the assessment. The result of the selections contained in each section, risk estimation, risk reduction, and characteristics of facility, clearly indicate if the existing conditions reduce the likelihood of an injury occurrence to a tolerable level and the likelihood of an injury occurrence.

By providing the user with pre-defined choices the risk estimation can be manipulated such that the responsibility for risk reduction measures can be distributed to either the original equipment manufacturer (OEM)/integrator or the facility that will utilize the equipment by simply reviewing the remaining level of risk that is shown at the bottom of each section. This action will assist the equipment designer or the equipment purchaser in managing the risks to which each company is exposed as well as help to manage equipment and manpower expenditures.

It is the manufacturer's responsibility to assess the user's specifications and to ensure that the usage of the machine, considering its capacity and limits with the work environment and the maintenance program, will satisfy the user's requirements. It is also the manufacturer's responsibility to warn the user if the requirement cannot be fulfilled, either totally or partially, and to provide a list of foreseeable residual risks in the latter case.

Risk Assessment

Risk Assessment, when reduced to its most basic format, is nothing more than an argument between the capabilities of a machine, A, and the abilities of an end user, B. This argument provides two parts of a three part equation, A+B=C. The correct answer to this equation is equal to C, the protective measures that will reduce potential risk to a level that can be tolerated by the employer in the form of financial liability and the end user in the form of a safe working environment.

Once the equation has been broken down into three parts, these parts can be subdivided into several unique sections. In the following pages you will find general instructions which will help you to populate the risk assessment form such that a reasonable result can be established.

Using the Program

Creating a Document

When the “Create Document” button is pressed a form appears that will require you to enter some information that will be associated with the equipment assessment. This information must be entered prior to completion of the assessment process. Required fields are as follows:

Document Name

The document name can be assigned using any naming convention you wish. It is important however to keep in mind that a structured naming convention could be useful at a later date when document searches are performed.

Example;

RA0000103

| RA | represents the file type (risk assessment) |
|---|---|
| 00001 | represents the document number (incremental) |
| 03 | represents the calendar year (2003) |

Client Name

The client name must be selected whenever a new document is created. If the client record does not yet exist pressing the button next to the selection list will cause a client creation form to appear. In this form all pertinent client information can be entered if the “add” button is pressed. Once the information has been entered, actuation of the update button will store the data and actuation of the return button will take the user back to the document form.

Equipment ID

A unique equipment identifier should be assigned to each piece of equipment within a facility. If the equipment record does not yet exist, pressing the button next to the selection list will cause an equipment creation form to appear. In this form, all pertinent equipment information can be entered if the “add” button is pressed. Once the information has been entered, actuation of the update button will store the data and actuation of the return button will take the user back to the document form.

The equipment identifier could include a group of machines that is operating as a cell or an individual machine. If a group of machines is being assessed it is suggested that all of the equipment serial numbers be documented in the notes section of the document form.

Date Created

This field is automatically populated with a date that represents when the document was originally created. This is not an editable field.

Document Supplied By

This field is automatically populated with the name of the company or person who is the registered user of the software program. This information is provided during the software installation process. This field cannot be edited.

Document Versions

The document version section of the program performs the revision control function of the risk assessment program. Prior to continuing with the risk assessment an original version must be created. This process is initiated by actuating the “Create Version” button. Actuating the “Create Version” button will cause the document version form to appear.

Document Version

Each document that is created using the program will default to “Document version”. This field cannot be edited.

Version Opened Date

The version opened date represents the date that the document version was created. This field is populated automatically and the date is used by the revision control module of the program.

Version Closed Date

The version close date is selectable by the user via the pull down calendar. Normally the close date is set far enough in the future that ample opportunity is provided to complete the assessment prior to the version being locked. The program will automatically protect the document version once the version close date is reached. No changes are permitted to the protected document.

Assessment Performed By

The assessment performed by field should contain a list of all of the assessment participants. Generally, risk assessments are performed by a team of people including Engineers, users, managers, and often legal council. The combined knowledge base of all of these people helps to provide a very thorough assessment of the machine performance expectations and the expected or foreseeable misuse of the equipment.

Assessment Approved By

Hazardous industrial machinery risk assessment is a process that should be completed by a knowledgeable group of people that have had a substantial amount of exposure to the type of machinery being examined. This information, by using this program, will be collected and stored in a usable format that will in the end provide conclusive statements in a formatted report. In many jurisdictions, the assessment will only be considered valid if it is approved and signed by a Professional Engineer. The printed report will also bear the stamp of the Engineer that has validated the report. The Engineer's name should be entered in this field. Industrial safety regulations of the local jurisdiction of the facility must be consulted.

Reason for New Version

Each new document that is created should be supported by documenting the reason for performing the assessment. This field will have a default value of “New Document” . Additional information can be added to this field if desired. Subsequent versions of the document should be supported with a valid reason for re-assessing the equipment. Reasons for a new version could include, but are not limited to, new regulations, additional cell components, new training procedures, new guarding installation or any other reason that could effect the performance of the safety measures that were originally documented.

Revision Control Using Document Versions

Creating an Assessment

Characteristics of equipment (OEM/Integrator/User Responsibility)

Activity

This editable cell should contain a description of the task that is expected to be performed, by the end user, in the hazardous area. It is imperative, when providing this information that the greatest consideration is given to the activity that would expose the end user to the most severe conditions. For example, an operator that works in close proximity to the hazard regularly for long periods of time, “parts removal”, obviously has a greater risk of being injured than an engineer who must walk past the machine on route to their office once daily. Casual exposure has not been included in the example list. However, if the engineer were to be the only person exposed to the hazard, “Office personnel walking past” would be an appropriate entry. Multiple entries are allowed in this section.

Examples:
Normal operation
Stocking/restocking
Set-up or changeover
Shut down
Parts removal
Parts replacement
Lubrication
Periodic maintenance
Special maintenance
Quality testing
Supervisory task(s)
Demonstration
Trouble-shooting/problem solving
Installation
Finishing task(s)
Clean up
   Point of Operation

This editable cell should contain a description of machine tool area in which the hazardous motion will occur. Each hazardous area, of the equipment that is being analyzed, must be documented on an individual basis. For this reason, the risk analysis conclusion for a given piece of equipment may contain several separate reports.

Examples;
Molding area
Milling head
Conveyor drive
Part take-off zone (robot)
Punch turret
Drive mechanism of palletizer
Material extruder
Servo drive belt
Material grinder
Break press die
Spot welder tips
Automated guard leading edge
   Hazardous Motion(s)

This editable cell should contain a description of the motion that is the cause of the potential injury. In order to produce a valid report, this part of the risk assessment should be completed by an assessment team. Involving people with experience, and exposure to a wide variety of industrial environments will help to minimize the chances of an oversight during the analysis.

Examples;
Platen motion
Pallet lowering
End of arm tooling movement
Part ejector movement
Part ejection
Material feed mechanism
Spindle rotation
Material clamping mechanism
   Description of hazard(s)

This editable cell should contain a description that describes the general nature of the hazard within the hazardous area described above and the foreseeable conditions that could lead to user injury.

Examples;
Impact due to robot arm motion during setup
Part ejector pinch points during part removal
Shock hazards due to live electrical parts during maintenance
Expulsion of pressurized liquids during flow adjustment.
Bum due to egress of molten material
Entanglement of hands in drive belt
Impact due to movement of automatic guard
   Risk Estimation

Now that the particular hazardous situation has been described you can begin to estimate the risk of injury to which the end user will be exposed. In this section, it is essential to make your choices within the realm of an actual industrial environment rather than an ideal controlled situation.

This section is a pure analysis of machine capability. Hazards are to be discovered by viewing the machine as if no guarding has been installed. Therefore all machine and end user motions must be considered.

Severity of Potential Injury Caused by Hazard

Minor—Reversible injuries include but are not limited to scrapes, bruises, small cuts and other injuries that if properly cared for will normally heal within 1 to 2 weeks. Immediate return to work is possible.

Moderate—Normally reversible injuries include but are not limited to deep flesh wounds, minor bone fracture, sprains, and other injuries that would normally require professional medical attention. Return to work performing the same task is possible within 3 days.

Serious—Normally reversible injuries include but are not limited to major bone fracture, deep cuts causing tendon or muscle damage, small first or second degree burns, and other injuries that would normally require professional medical attention. Return to work performing the same task is possible.

Major—Normally irreversible injuries include but are not limited to compound bone fractures, third degree burns, blindness, loss of appendages and other injuries that normally cannot be repaired well enough to achieve a level of functionality that would permit the injured party to return to work performing the same or an equal task.

Fatal—Immediate or subsequent death hazards include but are not limited to explosions, crushing forces where the head or whole body are exposed to the hazard, inhalation of poisonous vapors which have an immediate effect on the central nervous or respiratory system, and other injuries which require immediate emergency medical attention in order to have any chance of survival.

Frequency of Exposure to Hazard

If you are an OEM, you are expected to make your selection based on the worst case scenario for the machinery being assessed.

Infrequent—Weekly or less is normally used to describe fully automated production areas

Occasional—Daily is normally used to describe work areas that require some activity to be performed on a daily basis such as tool setup, mold cleaning, machine lubrication.

Frequent—More than twice daily is normally used to describe work areas that require access due to varying production requirements that cause frequent tool changes or maintenance functions.

Continuous exposure—Hourly is normally used to describe work areas that require manual part removal or close visual inspection of a process to be performed constantly.

Possibility of Hazard Avoidance

Possible—Easily able to avoid is normally used to describe hazardous motions that occur in plain view of the exposed person and occur at a speed of less than 125 mm/second.

Possible—Potentially able to avoid is normally used to describe hazardous motions that occur in plain view of the exposed person and occur at a speed of more than 125 mm/second but less than 250 mm/second.

Unlikely—Unable to avoid is normally used to describe hazardous motions that occur either in plain view of the exposed person and occur at a speed of more than 250 mm/second or not in plain view of the exposed person and occur at a speed of less than 250 mm/second.

Impossible—injury is unavoidable is normally used to describe hazardous motions that, regardless of the location of the hazard, moves at such a speed that the exposed person would have little or no opportunity to escape harm.

Risk Reduction Method

Now that the characteristics of the hazard have been determined and the likelihood of injury occurrence, without the employment of protective elements, has been established protective measures must be selected that will reduce the likelihood of a person being exposed to the area in which the hazard will occur. In this section the user is required to have a thorough understanding of the terms such as control reliable, interlocked, barriers, and the definitions of electrical designs commonly referred to as categories 1 through 4. These terms are defined in numerous machine safety guidelines. Failure to fully understand these terms or the type of circuitry with which they are associated could lead to an inappropriate risk assessment and therefore possibly an injury occurrence.

There are two separate “Primary safety element” selection lists. The first list, mechanical, includes risk reduction methods that are active in nature or solely dependent on personnel interaction. Types of interaction include visual recognition, physical access restriction, and providing access by the requirement of physical effort. The second selection list includes risk reduction methods that are passive in nature. This type of safety element will not prevent access to the hazardous area but will prevent the occurrence of the hazardous event if a person enters the hazardous area. Only one of the two primary safety elements may be selected for each point of operation. Note: The ejection of material from within the hazardous area will not be prevented if a presence sensing device is employed as the primary safety element.

This section also has the provision for the selection of an additional control reliable safety device. This selection can be applied to many different situations. This selection could include a hydraulic or pneumatic valve with an electrical interlock that is monitored in the engaged and disengaged position, a cycle initiation device that operates independent of the primary safety element, or any other group reliable control components that function independent of the primary safety element.

In order for this selection to be considered valid, the electrical circuitry, the components used, and the software supervision, must have equal or greater integrity than that of the safety device that is being bypassed. By providing an administrative control to bypass a safeguard, risk of injury, and the liability that would be generated should an injury occur due to the bypassed safety device is transferred from the OEM/Integrator to the employer of the end user.

Primary safety element employed to reduce hazardous exposure. (Mechanical)

Warning signs

Awareness Barriers

Perimeter Barriers

Fixed Guards

Moveable Guards (Monitored)

Moveable Guards (Single channel interlock)

Moveable Guards (Dual channel interlock/cross monitoring Cat 3)

Moveable Guards (Dual channel interlock/cross monitoring Cat 4)

Moveable Guards (Dual channel interlock/cross monitoring Cat 4)

Moveable Guards w/guard locking (Dual channel interlock/cross monitoring Cat 3)

Primary safety element employed to reduce hazardous exposure. (presence sensing)

Light curtain/Safety mat (monitored)

Light curtain/Safety mat (Single channel interlock/monitored Cat 2)

Light curtain/Safety mat (Dual channel interlock/cross monitoring Cat 3)

Light curtain/Safety mat (Dual channel interlock/cross monitoring Cat 4)

Characteristics of Facility (Employer Responsibility)

Nature of Person Exposed to Hazardous Area

Employing personnel who have acquired a complete understanding of both the equipment being used and the activity being performed can greatly reduce the risk of injury. Often, this is not a practical approach to risk reduction due to the continuing cost of employing personnel who have achieved a very high level of qualification. Adequate safeguarding, on the other hand, often requires more capital investment. This, however, is an anticipated expense that will not be affected by employee availability conditions in the future.

The employee hiring practices of a facility must always take into consideration hazardous conditions that could exist in the facility. Once the risk assessment report has been completed, employee hiring policies should be review in order to ensure that past hiring practices have not placed current employees in a high risk situation.

Third Party Casual Exposure

This selection is used to describe people who are not directly employed by the facility. The people that should be considered to be part of the group could be sales people, customers, vendors, cleaning staff, or any other person that would not normally be exposed to a hazardous industrial environment.

Operator

This selection is used to describe people who are employed by the facility, whose primary responsibility is day-to-day machine operation. They could be performing a wide variety of tasks, all of which should be product oriented. It is quite common for machine operators to perform some machine maintenance tasks. For the purpose of assessing risk, the nature of the exposed person should be based on their job description rather than their task list.

Local Maintenance/Set-Up

This selection is used to describe people who are employed by the facility, whose primary responsibility is day-to-day activities that are more closely related to the production process and the machine condition than the final product handling. These people generally do not operate a single machine, but are exposed to many different machines within the facility. They are commonly exposed to much more hazardous conditions than the operator or a third party person who is casually exposed person.

Third Party Service/Repair

This selection is used to describe people, who are not directly employed by the facility, whose primary responsibility is to provide support to the facility under contract conditions. The third party is responsible for the training and knowledge of the person that they employ. The facility however must state the service person's minimum level of qualification in the service contract and it must be made clear in the contract that the safety procedures and policies of the facility apply to all persons who enter the facility. Generally, service or repair technicians must have attained some form of trade certification.

Qualification/Education Level of the Person Exposed to Hazardous Area

Accuracy is of utmost importance in this selection. Failure to state the actual qualification of the exposed person could lead to an injury occurrence. All employee qualifications should be supported by official documentation.

No Knowledge of Hazardous Environment

This selection applies to a person that has never received any training and has not been exposed to a set of conditions similar to the hazardous area that is being assessed. This selection generally applies to people who have little or no experience in an industrial environment.

Previous Exposure in Similar Environment (No Formal Training)

This selection applies to a person that has never received any training but has been exposed to a set of conditions similar to the hazardous area that is being assessed. Generally, a person who has worked in a similar environment for a period of time equal to or greater than 6 months will have become aware of some industrial hazards and how to avoid injury. Although experience is a valuable asset it can rarely replace formal training.

Previous Exposure in Similar Environment (Formal Employee Training)

This selection applies to a person that has received formal employee training and has been exposed to a set of conditions similar to the hazardous area that is being assessed. Generally, a person who has worked in a similar environment for a period of time equal to or greater than 6 months will have become aware of some industrial hazards and how to avoid injury. Work experience, when coupled with formal employee training, will drastically reduce the frequency of an injury occurrence. In many facilities, an employee of the facility provides training. Although this situation provides the advantage of the trainer's experience within a particular area, it may not provide sufficient information regarding government regulations or industry guidelines.

Previous Exposure in Similar Environment (Formal Training Provided by Professional Trainer)

Generally, a person who has worked in a similar environment for a period of time equal to or greater than 6 months, will have become aware of some industrial hazards and how to avoid injury. Work experience, when coupled with professional formal employee training, will drastically reduce the frequency of an injury occurrence. Third party involvement in employee training provides the transfer of knowledge that is gathered from a wide range of industrial conditions, and the exposure to many of the safety regulations under which industrial establishments are expected to operate. Professional trainers must be able to qualify their credentials. Normally, they are required to have received training from other accredited sources and have to have regularly attended refresher seminars in order to maintain the status as a professional trainer. Submission of documentation supporting these qualifications should be requested prior to granting a training contract.

Previous Exposure in Similar Environment (Government Certified Training Certificate)

This is often the highest level of training that has commonly been achieved by an employee. This type of certification is normally applicable to employees who have completed an apprenticeship program in a specific trade or occupation. This type of certification is normally coupled with several years of work experience in the related occupation.

Personal Protective Equipment (PPE) Determined by Task Hazard Assessment

In order to properly select personal protective equipment, task assessment and hazard identification must be completed. This process includes the identification of all actions the employee is expected to perform and to which hazards the will be exposed.

For example;

An employee, who is expected to manually remove sheet metal parts from a turret punch press, would regularly be exposed to high levels of noise, potential falling material, sharp edges, and potential ejection of small pieces of sheet metal. For this situation, the use of safety certified footwear, safety glasses, gloves, and hearing protection would be appropriate.

Many types of personal protective equipment must be certified by a nationally recognized testing laboratory such as UL or CSA. Local or regional regulations should be considered prior to using personal protective equipment in order to ensure sufficient protection is provided.

There are numerous combinations of PPE available. Only basic selections have been made available in the selection lists. We suggest that selection be made that most closely represents the PPE measures that are required at a specific point of operation. Additional information can be entered in the notes section of the risk assessment. The most important point is that task hazard analysis has been performed and appropriate PPE has been assigned.

Safety Certified Footwear, Safety Glasses

Safety certified footwear must be of such construction that it will provide adequate protection against the expected hazard. For example safety shoes do not provide complete ankle coverage therefore would be inappropriate protection where there is a possibility of the ankle being injured by falling debris.

Safety glasses must be of such construction that it will provide adequate protection against the expected hazard. Protective eyewear that does not provide protection from welding flash would be inappropriate in a welding booth.

Safety Certified Footwear, Safety Glasses, Gloves, Hearing Protection

In addition to the information provided above hearing protection that provides suitable protection for the noise levels in is required. In order to select the proper type of hearing protection normal and maximum decibel samples must be documented. The supplier of the hearing protection device will require this information in order to provide the proper protection device.

Gloves are also commonly used as a protective measure against minor abrasions, cuts. and burns etc. on hands and lower arms. Gloves should be of such construction that an appropriate level of protection would be achieved.

Safety Certified Footwear, Face Shield, Gloves, Hearing Protection

This selection replaces the safety glasses requirement shown above with a full-face shield. Full facial protection is normally required if there is potentially flying materials that are capable of causing an injury. In an arc welding environment, for example, flying sparks are a common hazard that could burn exposed flesh immediately upon contact. Full facial protection should be required under these conditions.

Safety Certified Footwear, Face Shield, Gloves, Coveralls, Hearing Protection

The addition of coveralls to the PPE selection normally indicates that exposure of skin in the environment could lead to an injury occurrence. Care must be taken in the selection of materials used in the construction of the coveralls to ensure employee comfort and mobility.

Safety Certified Footwear, Face Shield, Gloves, Coveralls, Protective Headgear, Hearing Protection

The addition of protective head gear to the PPE selection could be used if hazards that could cause injuries to the top, back or front of the head. These hazards are usually caused by, but not limited to, low ceiling height or falling objects.

Work Place Safety Policy

No Procedural Guidelines

This selection is used in a facility that does not provide any guidance to its employees. A facility of this nature normally has a wide variety of varying activities for which procedural guidelines are not considered. This situation is not considered acceptable in an industrial environment.

Informal Procedural Guidelines/Verbal Instruction

This selection is used in a facility that has given some consideration to task procedures performed in the facility. Instruction is provided verbally and the majority of the information is based on the past experience of the person providing the instruction. Verbal instruction is generally provided during initial employee training and is not documented.

Formal Procedural Guidelines for Some Activities/Verbal Instruction

Formal procedural guidelines are documents that are created and maintained at the facility. Initially guidelines for complex tasks during which the employee may encounter a hazardous situation should be created. These documents must have been made available to the employees. Verbal instruction, based on the procedural guidelines, is provided to the employee.

Safety Committee Guidelines for Some Activities/Written Instruction

Formal procedural guidelines are documents that are created and maintained at the facility. Initially guidelines for complex tasks during which the employee may encounter a hazardous situation should be created. These documents must have been provided to the employees prior to attempting a given task. Verbal instruction, based on the procedural guidelines, is provided to the employee.

Safety Committee Guidelines for All Activities/Written Instruction

Formal procedural guidelines are documents that are created and maintained at the facility. Assessments for all tasks during which the employee may encounter a hazardous situation have been created. These documents must have been provided to the employees prior to attempting a given task. Verbal instruction, based on the procedural guidelines, is provided to the employee.

Safety Committee Guidelines for All Activities/Written Instruction/Verbal Warning for Infraction

Formal procedural guidelines are documents that are created and maintained at the facility. Assessments for all tasks during which the employee may encounter a hazardous situation have been created. These documents must have been provided to the employees prior to attempting a given task. Verbal instruction, based on the procedural guidelines, is provided to the employee. The supervisor of the employee is held accountable for the safety of his or her subordinate and is actively supported by the facility when safety infractions occur. By providing verbal warning to the person who is in noncompliance with safety procedures many reoccurrences of unsafe work habits can be avoided.

Safety Committee Guidelines for All Activities/Written Instruction/Written Warning for Infraction

Formal procedural guidelines are documents that are created and maintained at the facility. Assessments for all tasks during which the employee may encounter a hazardous situation have been created. These documents must have been provided to the employees prior to attempting a given task. Verbal instruction, based on the procedural guidelines, is provided to the employee. The supervisor of the employee is held accountable for the safety of his or her subordinate and is actively supported by the facility when safety infractions occur. By providing written warning to the person who is in noncompliance with safety procedures many reoccurrences of unsafe work habits can be avoided. The employee is made aware that safety policies are in place and that the facility expects employees to abide by them.

Safety Committee Guidelines for All Activities/Written Instruction/Incremental Penalties for Infraction

Formal procedural guidelines are documents that are created and maintained at the facility. Assessments for all tasks during which the employee may encounter a hazardous situation have been created. These documents must have been provided to the employees prior to attempting a given task. Verbal instruction, based on the procedural guidelines, is provided to the employee. The supervisor of the employee is held accountable for the safety of his or her subordinate and is actively supported by the facility when safety infractions occur. By providing first a verbal and then written warning to the person who is in noncompliance with safety procedures many reoccurrences of unsafe work habits can be avoided. If safety infraction continue to occur notification of temporary suspension of employment is provided. Further infractions of facility safety policy can eventually lead to permanent employee termination if the facility feels that the repeat offender is placing the viability of the operation at risk. The employee is made aware that safety policies are in place and that the facility expects employees to abide by them or risk permanent termination.

Referring to Example No. 1, it can be seen that the risk estimation section relates solely to the machine or equipment being evaluated. Hazards of the particular machine are discovered by viewing the machine as if no guarding has been installed. There are three risk factors for risk estimation. These are severity of potential injury caused by hazard, frequency of exposure to the hazard and possibility of hazard avoidance. Each of these risk factors has a range of inputs, for example, the severity of potential injury caused by hazard has a range of input that are Minor, Moderate, Serious, Major and Fatal. As can be seen from Example No. 1, all of the inputs are further defined and, preferably are further defined on the electronic form itself so that a person conducting the evaluation has the information readily available in order to make an objective determination as to which input should apply to the particular hazard on the machine being evaluated. Similarly, the second risk factor under risk estimation is frequency of exposure to hazard. That risk factor also has a range of inputs as does the third risk factor, possibility of hazard avoidance. Preferably, there is a fourth risk factor that is part of the risk estimation, being probability of hazard occurrence.

Risk reduction is then considered for the particular machine or area of the machine that is being evaluated. With risk reduction, a primary safety element(s) is/are selected from a list of mechanical or presence sensing elements. The system permits the selection of either a mechanical safety element or a presence sensing safety element for each hazardous area-being investigated. The system will not allow both a mechanical safety element(s) and a presence sensing safety element(s) to be selected for the same hazardous area.

Risk reduction also includes an additional safety element as well as safeguard bypass with an administrative control and residual risks.

After the risk reduction is considered for the industrial equipment, the characteristics of the facility in which the equipment is to be operated are considered. Factors such as the qualifications for the type of person that will be exposed to the equipment. These would include third parties, operators, in-house maintenance personnel and maintenance personnel from off site. The qualification and education level of the persons exposed to the equipment is also considered. A broad range of inputs for this risk factor are available with respect to the training level or lack of training of the personnel. Another risk factor is the personal protective equipment that is worn by persons who are exposed to the industrial machines or equipment. A range of inputs are available for this risk factor. The Workplace Safety Policy of the facility is another risk factor for which a range of inputs are available.

After all of the required and optional inputs have been entered into the system, the system can be directed to determine or can automatically determine a level of risk for the industrial equipment being evaluated. The level of risk will provide the evaluator with a determination as to whether or not the risk of operating that industrial equipment is acceptable or unacceptable based on the inputs that have been made into the system in relation to that equipment. The system has predetermined values that are assigned to each input that is selected. The assessment of risk obtained for a particular piece of industrial equipment is determined by having the system automatically add up all of the inputs for risk estimation, all of the inputs for risk reduction and all of the inputs for characteristics of the facility.

Before using the system, the user must determine each hazardous point of operation of a particular machine or piece of equipment. For example, a machine may have more than one hazardous point of operation. An assessment of risk must be conducted using the system for each point of hazardous operation. The evaluator must describe the hazardous point of operation that is being evaluated in a particular instance so that when a report is produced by the system, it is a relatively simple matter for anyone who reviews the report subsequently to identify the point of hazardous operation that has been evaluated.

Each risk factor has a range of inputs from low risk to high risk. The number of inputs for each risk factor will vary with a minimum of two. Typically, there will be four to five inputs for each risk factor, but a risk factor can have ten or even more inputs. Each input is assigned a value based on the degree of risk attributed to that particular input. The relationship between the various inputs for one risk factor is usually non-linear. In other words, if there are five inputs, and the lowest input has a value of two, the highest input will often have a value much greater than ten. For some risk factors, the relationship between the lowest risk input and the highest risk input is linear. Further, when considering risk reduction, the assigned values are negative relative to the assigned values for risk estimate, which are positive. In other words, the total value of risk reduction is deducted from the total value for risk estimate. Preferably, values that increase risk are positive and values that decrease risk are negative. For risk factors relating to the characteristics of the facility, some of the inputs will be positive and some of them will be negative. For some risk factors, more than one input can be made. For example, more than one safety factor may be present.

The system of the present invention allows industrial equipment that has already been valuated using the system to be re-valuated any number of times. Each valuation can be saved electronically so that the history of risk assessment for a particular piece of equipment is always available. The system also allows photographs and other documentation, including a summary of incidents, to be loaded into the system and saved with respect to a particular file, as desired. If a piece of industrial equipment is moved to a new location within the same plant or moved to another plant, a re-assessment will or may be necessary. Further, the method of operation of the equipment or the tools that are used with the equipment may change or the qualifications of the operation may change. Each change requires a new assessment of risk.

In a further embodiment of the invention, instead of having a probability of hazard occurrence set inherently within the program at 100%, the system has inputs for a probability of hazard occurrence. Preferably, the inputs are over a broad range. The range of inputs can, for example, range from substantially 90 to 100% or from substantially 80 to 100%. Still more preferably, the range of inputs for a probability of hazard occurrence is from substantially 50 to 100% or from 0 to 100%.

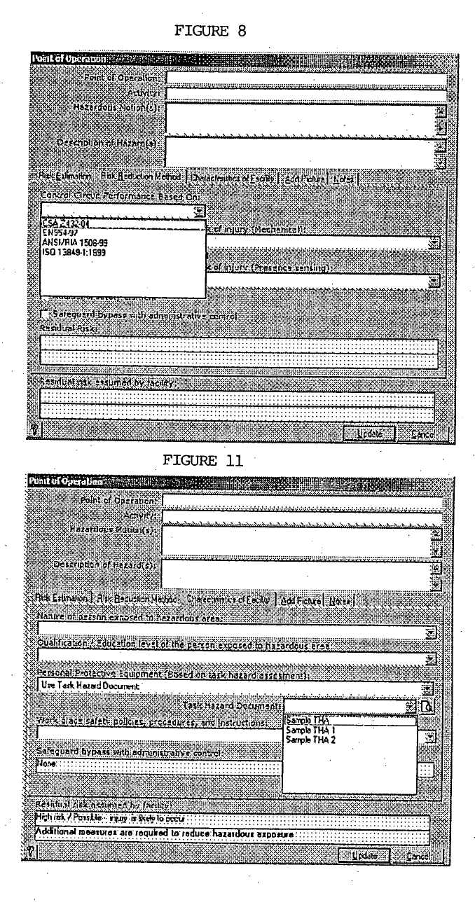

In FIG. 8, there is shown a drop down menu list for a safeguard standard (referred to as “control circuit performance based on:” in FIG. 8. The listing in FIG. 8 includes a choice of any one of four performance standards. Numerous variations can be made to the listing of performance standards. Preferably, the list includes at least one performance standard that is applicable to the jurisdiction in which the system is being used. By way of example, the list can include CSA Z432-04; EN954-97; ANSI/RIA1506-99; ISO 13849-1:1999, and CSA Z434-03. These standards are constantly being upgraded, revised and replaced. The references to CSA are Canadian Standards Association. It is important that the risk assessment be carried out with a standard that is acceptable and recognizable in the jurisdiction in which the assessment is being performed. The performance standards are constantly being updated, revised and/or replaced as continuous improvement to the standards is sought.

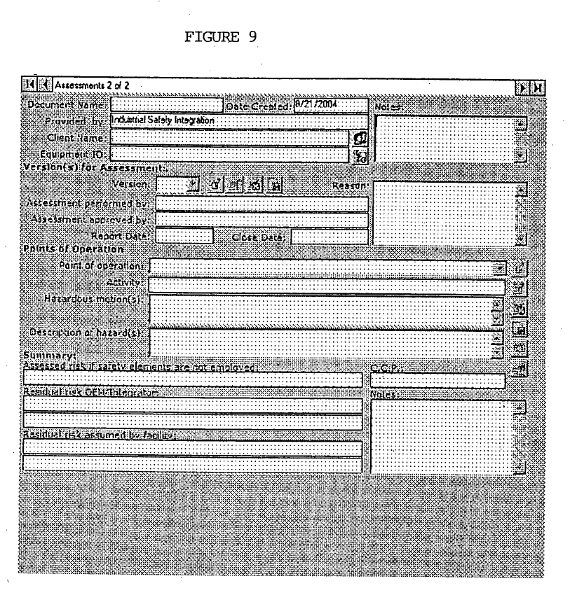

In FIG. 9, there is shown a top view of a form of assessment. It can be seen that the form has a space entitled CCP (Control Circuit Performance). When a safeguard performance standard is inserted into the form shown in FIG. 8, the standard chosen is automatically inserted into the CCP space in FIG. 9.

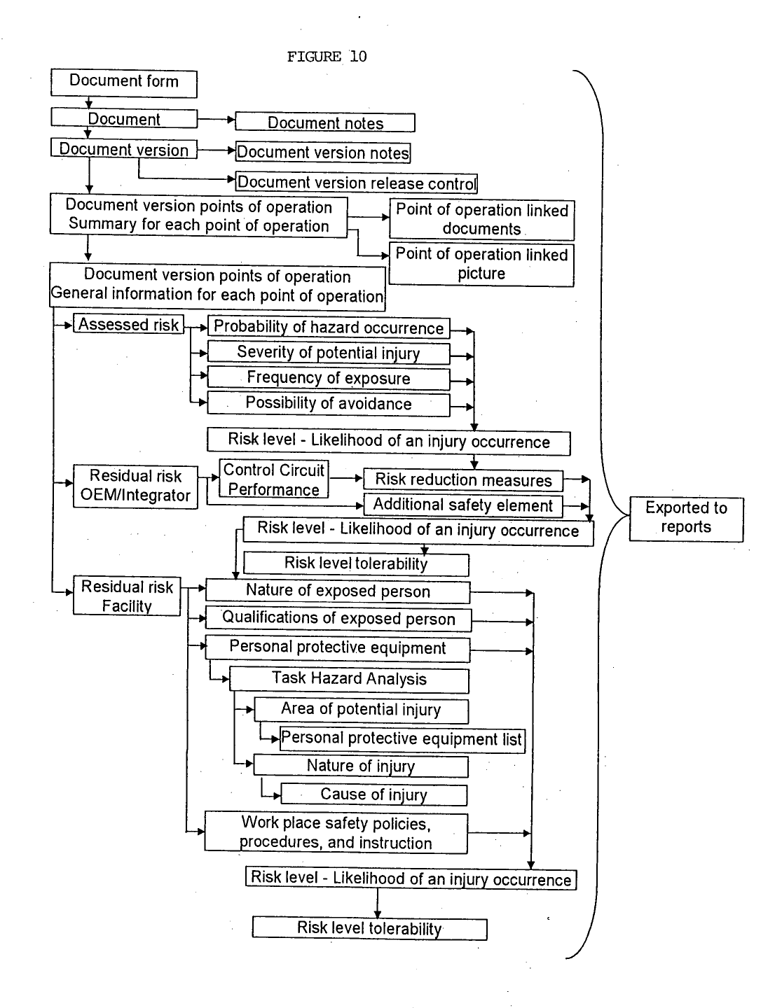

In FIG. 10, there is shown a block diagram for the risk assessment system of the present invention when that system includes a choice of safeguard performance standard. The block diagram is very similar to the block diagram shown in FIG. 2. However, a control circuit performance block has been inserted between the residual risk OEM/integrator and the risk reduction measures to allow a list of performance standards to be included from which a particular performance standard can be chosen for each point of operation. The performance standards chosen can be identical for all of the points of operation of a piece of equipment. The performance standards chosen can also be different between one point of operation and another point of operation for a piece of equipment. In addition to the choice of performance standard, the system shown in FIG. 10 allows an end user of the system to elect between using the drop down list for the personal protective equipment or starting with a blank page and inputting a list created by the end user for the personal protective equipment. In other words, the personal protective equipment list in the task hazard analysis portion of the system can be customized to the specific circumstances of the end user. The system recognizes inputs made to the revised list by the end user. When the user decides to use an edited list, the pre-determined list of protective equipment is no longer available to the user. The list created by the user can be revised after it is created. Alternatively, if the user wishes to revert to the predetermined list from the edited list, the user can do so.

In FIG. 11, there is shown a drop down space containing the words sample THA, sample THA1 and sample THA2. These samples are in a drop down space for a task hazard document. For each of these samples, an end user of the risk assessment system can insert any text that the user wishes to insert which would then become the task hazard document. The samples are examples only and further text can be inserted. The system is designed to recognize the edits. For example, if an end user wishes to add personal protective equipment to the equipment that is now set out in the predetermined drop down list, the end user is able to opt to edit and can then create the user's own list some of which may include all or part of the predetermined list. In other words, the end user can make a revised list of protective equipment or, alternatively, if the user has edited the system by adding text for task hazard measures, the end user can further edit the system by revising the edits that the end user had inserted previously.

Therefore, the end user has the ability to customize the program to suit the specific needs and requirements of the end user. The system does not permit the end user to revise or remove task hazard measures that are included in the drop down menus or pre-determined lists of the program. The end user can edit the program by adding input options to a new page through the text editing from the end user and having the system recognize the options. The input options relate to task hazards and, more particularly, to personal protective equipment. The new page can include task hazards from the pre-determined lists as the end user can insert those task hazards onto the new page along with any other task hazards that the end user wishes to add.

While the method and system of the present invention has been described in detail, numerous variations will be readily apparent to those'skilled in the art within the scope of the attached claims. The person who is using the method and system of the present invention should have a sufficient skill level to understand the various inputs available and to make realistic choices of the inputs that are selected.

1. A method of assessing the risk of using industrial equipment to a user thereof by preparing a risk evaluation using a program, said method comprising:
(a) choosing a safeguard performance standard for said risk evaluation;
(b) inputting to said program information relating to a plurality of risk factors;
(c) causing said program to estimate a net risk of injury to said user of said industrial equipment based on said information and based on evaluation data within said program;
(d) said program producing a risk evaluation for said industrial equipment;

2. A method as claimed in claim 1 including the step of choosing said performance standard from a pre-determined list of performance standards.

3. A method as claimed in claim 2 wherein said industrial equipment has a plurality of points of operation and said method includes the step of choosing one safeguard performance standard for each point of operation.

4. A method as claimed in claim 3 wherein said method includes the step of choosing a safeguard performance standard for each point of operation where the performance standard for one point of operation of said plurality of operations is different from the performance standard for another point of operation of said points of operation.

5. A method as claimed in claim 3 including the step of choosing at least one of said performance standards from a group of CSA, ANSI/RIA and ISO standards

6. A method as claimed in claim 3 including the step of choosing at least one performance standard from the group of CSA Z432-04, EN954-97, ANSI/RIA 1506-99, ISO 13849-1: 1999 and CSA Z434-03.

7. A method as claimed in claim 1 wherein one of said plurality of risk factors is based on safety characteristics of a particular facility in which said industrial equipment is to be used, said method including the step of estimating a level of risk reduction based on safety characteristics of said particular facility.

8. A method as claimed in claim 7 including the step of repeating the method for various pieces of industrial equipment, the risk evaluation being produced automatically.

9. A method as claimed in claim 7 including the steps of enabling an end user to edit said program by adding input options through text editing from said end user and having said system recognize said options.

10. A method as claimed in claim 7 wherein said program has inputs relating to task hazards, said method including the step of adding said input options to said task hazards in said program.

11. A method as claimed in claim 10 including the step of revising said input options.

12. A method as claimed in claim 10 including the step of adding said input options in relation to personal protective equipment.

13. A method as claimed in claim 7 including the step of inputting information of risk factors that increase risk together with risk factors that reduce risk.

14. A method as claimed in claim 13 including the steps of inputting information by estimating a risk of injury to said user based upon characteristics of said industrial equipment and estimating a level of risk reduction based upon safety features for said industrial equipment.

15. A method as claimed in claim 14 including the steps of the inputting to a program information relating to a plurality of risk factors by operating said program to produce several factors, each risk factor for which an input is available having a range of risk, selecting a level of risk from said range of risk for each risk factor that is applicable to said industrial equipment, said program estimating a net risk of injury to said user based upon said selections.

16. A risk assessment system for use with a computer, said system assessing the risk of injury to a user from industrial equipment by preparing a risk evaluation, said system comprising:
(a) a range of pre-determined risk values for each of a plurality of potential risk factors for which inputs are available for said industrial equipment;
(b) said system displaying each of said risk factors on demand and a range of inputs for a level of risk for each of said risk factors for which inputs are available;
(c) said system accepting an input for each risk factor for which inputs are available;
(d) said system determining a net value for all of said inputs and producing an assessment of risk for said industrial equipment;
(e) a selection for at least one safeguard performance standard for said risk evaluation.

17. A risk assessment system as claimed in claim 16 wherein said performance standard is available from a pre-determined list of performance standards within said program.

18. A risk assessment system as claimed in claim 17 wherein said industrial equipment has a plurality of points of operation and each point of operation has a safeguard performance standard.

19. A risk assessment system as claimed in claim 17 wherein said performance standard for one point of operation of said plurality of operations is different from a performance standard for another point of operation of said points of operation.

20. A risk assessment system as claimed in claim 16 wherein said performance standards are selected from a pre-determined list including at least one standard that is applicable in a jurisdiction where said user carries on business.

21. A risk assessment system as claimed in claim 16 wherein one of said risk factors is a level of risk reduction based on safety characteristics of a particular facility in which said industrial equipment is to be used.

22. A risk assessment system as claimed in claim 16 wherein said system produces a report setting out a risk evaluation for said industrial equipment, said system being set up to automatically determine a net value for all of said inputs and produce and assessment of risk for said industrial equipment.

23. A risk assessment system as claimed in claim 16 wherein said report is a printable report and includes said performance standard.

24. A risk assessment system as claimed in claim 18 wherein the performance standards for each point of operation are selected from the group of CSA Z432-04, End954-97, ANSI/RIA 1506-99, ISO 13849-1: 1999 and CSA Z434-03.

25. A risk assessment system as claimed in claim 16 wherein said system has an end user editing feature that allows said end user to edit said program by adding input options through text editing, said program recognizing said options.

26. A risk assessment system wherein said input options relate to task hazards.

27. A risk assessment system for use with a computer, said system assessing the risk of injury to a user from industrial equipment by preparing a risk evaluation, said system comprising:
(a) a range of pre-determined risk values for each of a plurality of potential risk factors for which inputs are available for said industrial equipment;
(b) said system displaying each of said risk factors on demand and a range of inputs for a level of risk for each of said risk factors for which inputs are available;
(c) said system accepting an input for each risk factor for which inputs are available;
(d) said system determining a net value for all of said inputs and producing an assessment of risk for said industrial equipment;
(e) at least one of a choice of a safeguard performance standard for said assessment and an end user editing feature that allows said end user to edit said program by adding input options through text editing, said system recognizing said options.
